(19) United States
(12) Patent Application Publication
(10) Pub. No.: US 2006/0142782 A1
(43) Pub. Date: Jun. 29, 2006

(54) Assembly for guiding insertion of a medical device into the brain's surrounding subdural space and method therefor

(76) Inventor: Daniel M. Lieberman, Phoenix, AZ (US)

Correspondence Address: WEISS & MOY PC, 4204 NORTH BROWN AVENUE, SCOTTSDALE, AZ, 85251, US

(21) Appl. No.: 11/024,213

(22) Filed: Dec. 28, 2004

(51) Int. Cl. A61F 11/00

(52) U.S. Cl. 606/108

(57) ABSTRACT

The assembly and method of the present invention guides the insertion of a medical device, such as a guide wire, into the subdural space surrounding the brain of a vertebrate. Preferably, the assembly is dimensioned to guide insertion of the medical device in a direction substantially tangent to the curvature of the brain. Further preferably, the assembly would allow its removal from the subdural space without disturbance of the location of the medical device. Still further preferably, the assembly will help to protect the brain from injury during insertion of the medical device into the subdural space.

### FIELD OF THE INVENTION

This invention pertains in general to devices and procedures for medical care and pertains in particular to medical devices and techniques for brain surgery.

### BACKGROUND OF THE INVENTION

As shown in FIGS. 5A-8, the human brain 124 is protected by the cranium 110, meninges, and cerebrospinal fluid (not shown). The cranium 110 is the brain's 124 bony enclosure. The meninges are the three tough, moist connective tissue membranes that surround the brain 124. From external to internal, the meninges consist of the dura mater 112, the arachnoid 116, and the pia mater 122. The narrow serous cavity between the dura mater 112 and the arachnoid 116 is the subdural space 114 and the wide space between the arachnoid 116 and the pia mater 122 is the subarachnoid space 118. The cerebrospinal fluid is the liquid found within the subarachnoid space 118.

A subdural hematoma is the accumulation of blood in the subdural space 114. Subdural hematomas typically result from traumatic head injuries that cause the rupture of blood vessels located within the meninges. One way of treating subdural hematomas is by evacuation using percutaneous drainage. Percutaneous catheter drainage has emerged as a less invasive technique for the treatment of chronic subdural hematomas, which may be performed at the patient's bedside. A catheter is inserted through a single burr hole 120 in the cranium 110 into the subdural space 114 and connected to a drainage bag (not shown). Using this technique, the subdural hematoma is drained slowly, over a period of days, allowing the brain 124 to gradually expand out toward the cranium 110 without creation of a new space, thereby reducing the risk of subdural hematoma recurrence.

In order to ensure that the catheter is properly positioned, a surgeon will typically use a guide wire to create a path into the subdural space 114. The surgeon first inserts the curved end of a J-shaped cannula through the burr hole 120 and into the subdural space 114. While holding the J-shaped cannula in place, the surgeon will then insert the guide wire through the cannula and into the subdural space 114. Once the guide wire is in place, the surgeon will remove the cannula by sliding it back along the length of the guide wire.

The equipment and the procedure described above present a great danger of injury to the brain. Specifically, guide wires are typically made of high tensile strength stainless steel. These stainless steel guide wires will not tolerate the strain of bending ninety degrees without plastically deforming and therefore retaining the curvature of the forced bend. The result of being forced to curve seventy to ninety degrees after entering the subdural space 114 through the J-shaped cannula causes the stainless steel guide wire to arc backward into the skull and may potentially cause undesired piercing trauma. Furthermore, a stainless steel guide wire which is forced to curve seventy to ninety degrees by the J-shaped cannula used to guide it, by the very nature of that force and the resultant friction between the stainless steel guide wire and the J-shaped cannula, resists the removal of the J-shaped cannula without the associated disturbance of the location of the stainless steel guide wire. Still further, the small diameter of the stainless steel guide wire may cause piercing trauma as it is inserted into the subdural space 114 even though rounded on its distal end.

Therefore, a need existed for an assembly and method for guiding insertion of a medical device, such as a guide wire, into the subdural space surrounding a brain. Preferably, the assembly will be dimensioned to allow its removal from the target area without disturbance of the location of the guide wire. Further preferably, the device would protect the brain from injury during insertion of the guide wire by directing the guide wire through the subdural space at an angle substantially tangent to the curvature of the brain.

### SUMMARY OF THE INVENTION

An object of the present invention is to provide an assembly for guiding insertion of a medical device, such as a guide wire, into the brain's surrounding subdural space.

It is a further object of the present invention to provide an assembly that may be removed from the target area without disturbance of the location of the guide wire.

It is a further object of the present invention to provide an assembly that would protect the brain from injury during insertion of a guide wire by directing the guide wire through the subdural space at an angle substantially tangent to the curvature of the brain.

### BRIEF DESCRIPTION OF THE PREFERRED EMBODIMENTS

In accordance with one embodiment of the present invention, an assembly for guiding insertion of a medical device into a target area of a vertebrate is disclosed. The assembly comprises: a first elongate member having a first end, a shaft portion coupled to and extending from the first end of the first elongate member, and a substantially C-shaped second end coupled to and extending from the shaft portion of the first elongate member and disposed opposite the first end of the first elongate member, the substantially C-shaped second end dimensioned to guide the medical device into the target area in a desired direction; and a second elongate member having a first end, a shaft portion defining a bore extending therethrough and the shaft portion being coupled to and extending from the first end of the second elongate member and being dimensioned to permit a medical device to be inserted therethrough, and a second end coupled to and extending from the shaft portion of the second elongate member and disposed opposite the first end of the second elongate member, the second end of the second elongate member dimensioned to be removably coupled to the substantially C-shaped second end of the first elongate member.

In accordance with another embodiment of the present invention, an assembly for guiding insertion of a medical device into a subdural space is disclosed. The assembly comprises: a first elongate member having a first end, a shaft portion coupled to and extending from the first end of the first elongate member, a neck portion coupled to and extending from the shaft portion of the first elongate member, a substantially C-shaped second end coupled to and extending from the neck portion of the first elongate member and disposed opposite the first end of the first elongate member, the substantially C-shaped second end dimensioned to guide the medical device into the subdural space in a direction substantially tangent to a curvature of a brain, and a groove defined by and extending along the substantially C-shaped second end of the first elongate member and dimensioned to permit a medical device to pass therethrough; a second elongate member having a first end, a shaft portion defining a bore extending therethrough and the shaft portion being coupled to and extending from the first end of the second elongate member and being dimensioned to permit a medical device to be inserted therethrough, a substantially half-cylindrical neck portion coupled to and extending from the shaft portion of the second elongate member, a substantially flat second end coupled to and extending from the substantially half-cylindrical neck portion of the second elongate member and disposed opposite the first end of the second elongate member, the substantially flat second end providing a roof for enclosing at least a portion of the groove defined by and extending along the substantially C-shaped second end of the first elongate member, and at least two clasps coupled to and curving inwardly from the roof of the substantially flat second end of the second elongate member, the clasps being dimensioned to engage the substantially C-shaped second end of the first elongate member; and a housing, defining a bore extending therethrough proximate a first side of the housing, the bore dimensioned to receive the shaft portion of the first elongate member and a groove extending along a second side of the housing, the groove dimensioned to snap-fit the shaft portion of the second elongate member wherein the housing being dimensioned to maintain the second elongate member at an approximately forty-five degree angle to the first elongate member.

In accordance with another embodiment of the present invention, an assembly for guiding insertion of a medical device into a subdural space is disclosed. The assembly comprises an elongate member having a first end, a shaft portion defining a bore extending therethrough and the shaft portion being coupled to and extending from the first end and being dimensioned to permit a medical device to be inserted therethrough, a neck portion coupled to and extending from the shaft portion, and a substantially C-shaped second end coupled to and extending from the neck portion and disposed opposite the first end, the substantially C-shaped second end dimensioned to guide the medical device into the subdural space in a direction substantially tangent to a curvature of a brain.

In accordance with yet another embodiment of the present invention, a method for guiding insertion of a medical device into a target area of a vertebrate is disclosed. The method comprises the steps of: providing an assembly having a first elongate member, a second elongate member, and a housing dimensioned to maintain the second elongate member at an approximately forty-five degree angle to the first elongate member; inserting a substantially C-shaped end of the first elongate member into the target area of the vertebrate; guiding the medical device substantially downwardly through a bore extending through the second elongate member; guiding the medical device along a groove defined by the substantially C-shaped end of the first elongate member; and inserting the medical device into the target area of the vertebrate in the desired direction.

In accordance with still another embodiment of the present invention, a method for guiding insertion of a super elastic nitinol guide wire into a subdural space is disclosed. The method comprises the steps of: providing an assembly having a first elongate member, a second elongate member, and a housing dimensioned to maintain the second elongate member at an approximately forty-five degree angle to the first elongate member; inserting a substantially C-shaped end of the first elongate member into the subdural space; guiding the super elastic nitinol guide wire substantially downwardly through a bore extending through the second elongate member; guiding the super elastic nitinol guide wire along a groove defined by the substantially C-shaped end of the first elongate member; inserting the super elastic nitinol guide wire into the subdural space in the direction substantially tangent to the curvature of the brain; detaching the second elongate member from the housing; sliding the second elongate member away from the target area while the super elastic nitinol guide wire passing therethrough while at the same time the super elastic nitinol guide wire maintaining its position within the subdural space; and removing the substantially C-shaped end of the first elongate member from the subdural space while at the same time the super elastic nitinol guide wire maintaining its position within the subdural space.

In accordance with still another embodiment of the present invention, a method for guiding insertion of a super elastic nitinol guide wire into a subdural space is disclosed. The method comprises the steps of: providing an elongate member having a first end, a shaft portion defining a bore extending therethrough and being dimensioned to permit a medical device to be inserted therethrough, a neck portion, and a substantially C-shaped second end defining a groove extending along the substantially C-shaped second end and dimensioned to permit a medical device to pass therethrough; inserting the substantially C-shaped second end into the subdural space; guiding the super elastic nitinol guide wire substantially downwardly through the bore; guiding the super elastic nitinol guide wire along the groove; inserting the super elastic nitinol guide wire into the subdural space in a direction substantially tangent to a curvature of a brain; removing the substantially C-shaped second end from the target area while the super elastic nitinol guide wire passing therethrough while at the same time the super elastic nitinol guide wire maintaining its position within the subdural space; and sliding the elongate member away from the target area while the super elastic nitinol guide wire passing therethrough while at the same time the super elastic nitinol guide wire maintaining its position within the subdural space.

The foregoing and other objects, features, and advantages of the invention will be apparent from the following, more particular, description of the preferred embodiments of the invention, as illustrated in the accompanying drawings.

### BRIEF DESCRIPTION OF THE DRAWINGS

FIG. 1 is a perspective view of one embodiment of the assembly of the present invention with a guide wire inserted therethrough.

FIG. 2 is a side, cross-sectional view of a substantially C-shaped second end of a first elongate member coupled to a second end of a second elongate member of the assembly of FIG. 1.

FIG. 3 is a perspective view of the substantially C-shaped second end of the first elongate member.

FIG. 4 is a perspective view of the second end of the second elongate member.

FIG. 5A is a perspective cross-sectional view of a brain and its protective meninges (dura mater, arachnoid, and pia mater), and cranium.

FIG. 5B is a side view of a guide wire being inserted through the assembly of FIG. 1 and into the subdural space.

FIG. 6 is a side view of the assembly of FIG. 1 with the first elongate member having been removed.

FIG. 7 is a side view of the guide wire with the first elongate member and the second elongate member having been removed.

FIG. 8 is a side view of a catheter being inserted into the subdural space by sliding it along the guide wire.

FIG. 9 is a cross-sectional view of the catheter and the guide wire of FIG. 8, taken along line 9-9.

FIG. 10 is a perspective view of another embodiment of the assembly of the present invention.

FIG. 11 is a side cross-sectional view of the assembly of FIG. 10 taken along line 11-11.

FIG. 12 is another embodiment of the assembly of the present invention.

FIG. 12A is a partial rear view of the assembly of FIG. 12 taken along line 12A.

FIG. 13 is a partial perspective view of yet another embodiment of the assembly of the present invention.

### DETAILED DESCRIPTION OF THE PREFERRED EMODIMENTS

The novel features believed characteristic of the invention are set forth in the appended claims. The invention will best be understood by reference to the following detailed description of illustrated embodiments when read in conjunction with the accompanying drawings, wherein like reference numerals and symbols represent like elements.

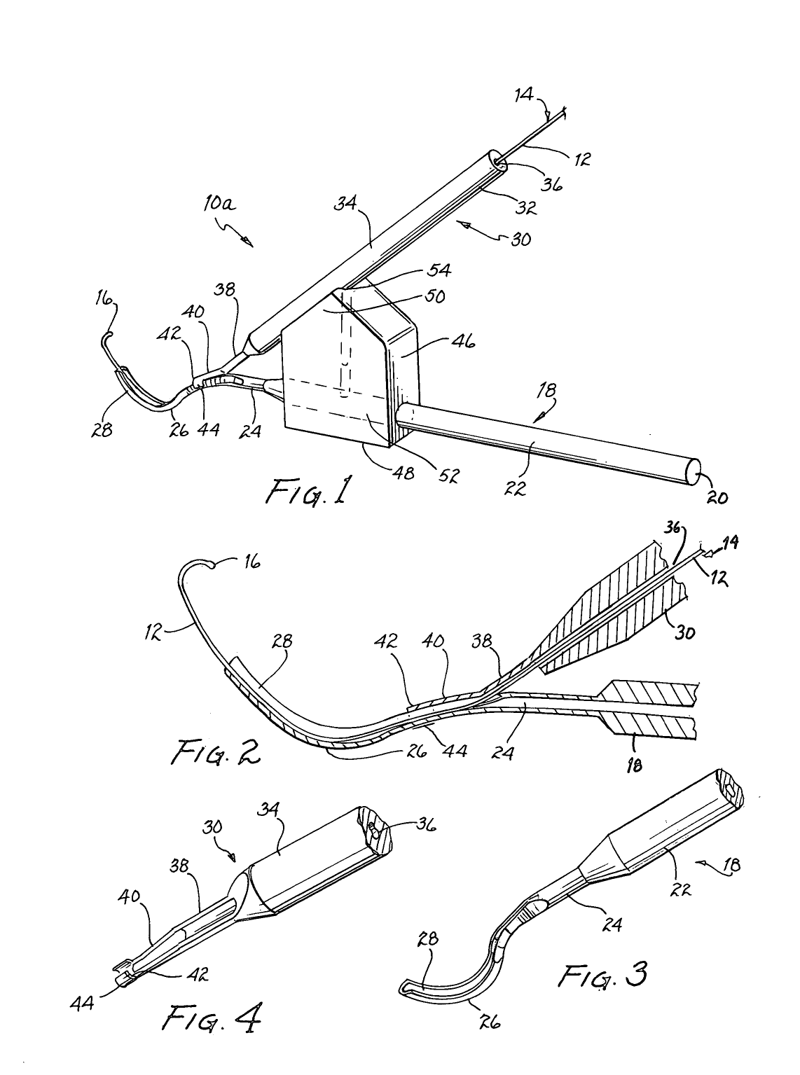

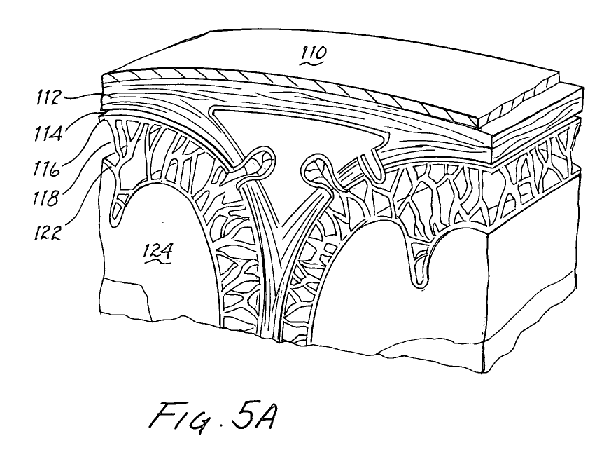

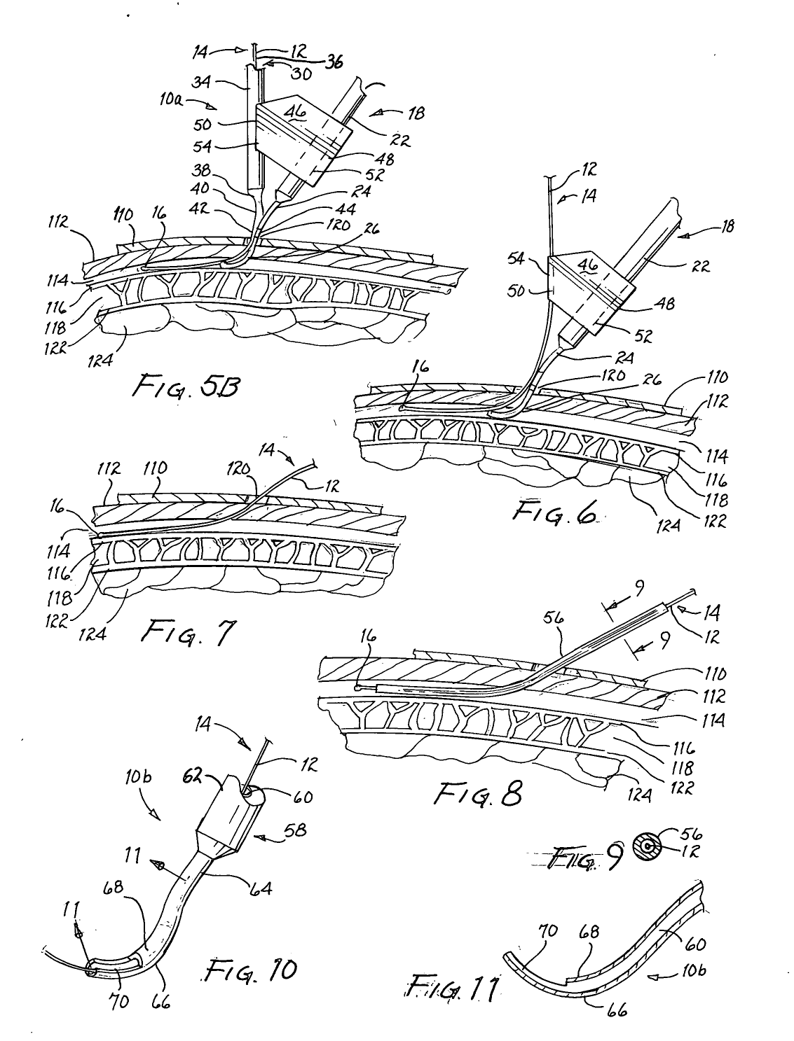

Referring to FIGS. 1-4, FIGS. 5B-6, FIGS. 10-11, and FIGS. 12-13, various embodiments of an assembly for guiding insertion of a medical device 14 into a target area of a vertebrate, referred to generically as an assembly 10, are disclosed (medical device 14 and assembly 10 are not shown on drawing FIGS. 3-4, assembly 10 is not shown in FIG. 6, and medical device 14 is not shown in FIG. 11). Preferably, the assembly 10 is dimensioned to guide the medical device 14 (see FIGS. 1, 2, 5B, 6, 7, 8, and 13), such as a guide wire 12 (see FIGS. 1, 2, 5B, 6, 7, 8, and 13), into the subdural space 114 (shown in FIGS. 5A . . . etc.) in a direction substantially tangent to the curvature of the brain 124 (shown in FIGS. 5A . . . etc.). Insertion in such a direction helps to prevent injury to the brain 124. Preferably, the guide wire 12 comprises a dull, substantially bulbous-shaped end 16 (see FIGS. 1, 2 and 5B-8) so as to protect the brain 124 from injury in the event that the guide wire 12 contacts the brain 124, however substantial benefit could be derived from an alternative configuration of the invention in which the guide wire 12 does not comprise a substantially bulbous-shaped end 16. Although the assembly 10 is preferably dimensioned to be used in surgery on human beings, it should be clearly understood that substantial benefit could be derived from using the assembly 10 on other mammals or vertebrates. It should be further understood that although it is preferred that the assembly 10 be used to guide insertion into the subdural space 114, substantial benefit may be derived from using the assembly to guide insertion into an alternate area of the brain 124.

As shown in FIGS. 1-4, 5B-6, and 12-13, the assembly 10a (assembly 10a is not shown on drawing FIGS. 3-4 or FIG. 6), referred to generically as assembly 10, comprises a first elongate member 18 (see FIGS. 1-3, 5B-6 and 12-13) and a second elongate member 30 (see FIGS. 1-2, 4, 5B-6 and 12-13). The first elongate member 18 and the second elongate member 30 are each preferably coupled to a housing 46 (see FIGS. 1, 5B, 6, 12, 12A, and 13). The housing 46 is dimensioned such that the first elongate member 18 and the second elongate member 30 form an angle of approximately forty-five degrees. Although forty-five degrees is the preferred angle, it should be clearly understood that substantial benefit may be derived from an alternate angle, so long as the angle is less than ninety degrees. While, in the preferred embodiment, the first elongate member 18 and the second elongate member 30 are coupled to a housing 46 it should be clearly understood that substantial benefit could be derived from an alternative embodiment of the present invention in which there is no housing 46. For example, it may be beneficial for the first elongate member 18 and the second elongate member 30 to be directly coupled to one another, or coupled in some other fashion that does not require a housing 46.

The first elongate member 18 preferably comprises a first end 20 (see FIGS. 1 and 13), a shaft portion 22 (see FIGS. 1, 3, 5B, 6, 12 and 13), a neck portion 24 (see FIGS. 1-3, 5B, 6, 12 and 13), and a substantially C-shaped second end 26 (see FIGS. 1-3, 5B-6 and 12). As shown in FIGS. 1-3 and 12, the substantially C-shaped second end 26 defines a groove 28 extending along the substantially C-shaped second end 26. The groove 28 aids in guiding the guide wire 12 into the subdural space 114.

The second elongate member 30 (shown in FIGS. 1, 2, 4, 5B, 12, and 13) preferably comprises a first end 32 (see FIGS. 1 and 13 (first end 32 is not shown in FIG. 13)), a shaft portion 34 (see FIGS. 1, 4, 5B, 12, and 13), a substantially half-cylindrical neck portion 38 (see FIGS. 1, 2, 4, 5B, 12 and 13), and a substantially flat second end 40 (see FIGS. 1, 2, 4, 5B, and 12). The shaft portion 34 defines a bore 36 (see FIGS. 1, 2, 4, 5B, and 13) extending along the full length of the shaft portion 34 and the bore 36 is dimensioned to allow a guide wire 12 to be inserted therethrough. As shown in FIGS. 1, 2, 4, 5B, and 12, the substantially flat second end 40 preferably provides a roof 42 for enclosing at least a portion of the groove 28 defined by the substantially C-shaped second end 26 of the first elongate member 18. And as shown in FIGS. 1, 2, 4, 5B, and 12, two clasps 44 preferably curve substantially inwardly from the roof 42 and are dimensioned to engage the substantially C-shaped second end 26 of the first elongate member 18. While, in one embodiment of the present invention, the assembly 10 comprises a first elongate member 18 having a neck portion 24, a substantially C-shaped second end 26 defining a groove 28, it should be clearly understood that substantial benefit could be derived from an alternative embodiment of the present invention in which the first elongate member 18 is alternatively configured so long as the first elongate member 18 is capable of guiding a guide wire 12 into the subdural space 114. Furthermore, while in one embodiment of the present invention the second elongate member 30 comprises a substantially half-cylindrical neck portion 38, a substantially flat second end 40 that provides a roof 42 for enclosing at least a portion of the groove 28 defined by the substantially C-shaped second end 26 of the first elongate member 18, and two clasps 44 dimensioned to engage the substantially C-shaped second end 26 of the first elongate member 18 it should be clearly understood that substantial benefit could be derived from an alternative embodiment of the present invention in which the second elongate member 30 is alternatively configured so long as the second elongate member 30 is capable of assisting the placement of a guide wire 12 into the subdural space 114.

Referring to FIGS. 1, 5B, and 6, the housing 46 may have one bore 52 extending therethrough along a first side 48 of the housing 46. As shown, the bore 52 is dimensioned to receive the shaft portion 22 of the first elongate member 18. The housing 46 may also have one groove 54 extending along a second side 50 of the housing 46. As shown, the groove 54 is dimensioned to preferably snap-fit the shaft portion 34 of the second elongate member 30. Although the figures show the bore 52 along the first side 48 of the housing 46 and the groove 54 on the second side 50 of the housing 46, it should be clearly understood that substantial benefit may be derived from the bore 52 appearing along the second side 50 of the housing 46 and the groove 54 appearing along the first side 48 of the housing 46. While, in the preferred embodiment, the groove 54 is dimensioned to receive the shaft portion 34 by means of a snap-fit coupling it is also within the spirit and scope of this invention that the shaft portion 34 rest in the groove 54 without being coupled by means of a snap-fit.

Referring to FIG. 12, the housing 46 may alternatively have two grooves 54; one groove 54 extending along the first side 48 of the housing 46 and the other groove 54 extending along the second side 50 of the housing 46. Or, referring to FIG. 13, the housing 46 may have two bores 52 extending therethrough, one bore 52 extending along the first side 48 of the housing 46 and the other bore 52 extending along the second side 50 of the housing 46. Substantial benefit may be obtained from any of the above configurations of the housing 46, so long as the second elongate member 30 may be easily detached from the housing 46 and removed without disturbing the position of the guide wire 12 within the subdural space 114.

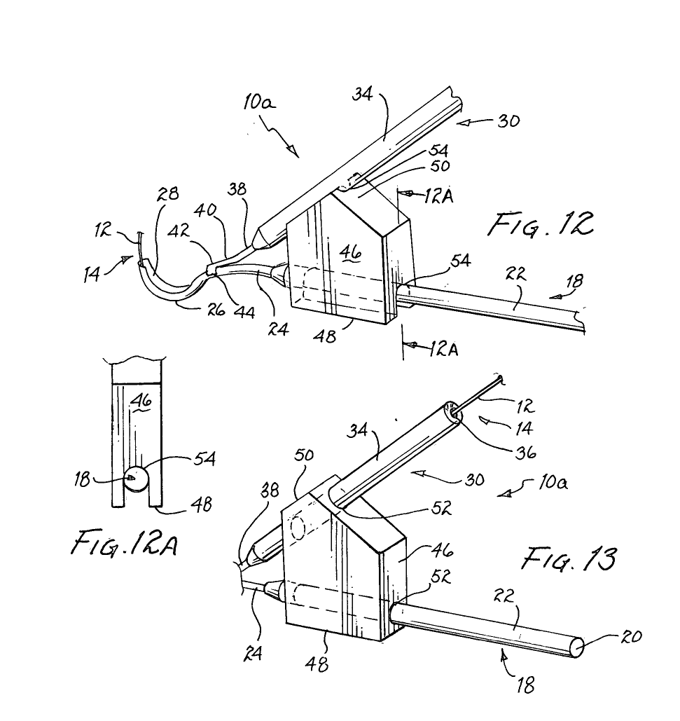

Referring now to FIGS. 10 and 11, an alternative embodiment of the assembly 10, referred to as assembly 10b, is shown. The assembly 10b, while serving the same purpose as assembly 10, comprises only one elongate member 58 (see FIG. 10) having a first end (not shown), a shaft portion 62 (see FIG. 10), a neck portion 64 (see FIG. 10), and a substantially C-shaped second end 66. The shaft portion 62 defines a bore 60 extending therethrough and is dimensioned to allow a guide wire 12 to be inserted therethrough. The substantially C-shaped second end 66 defines a groove 70 extending along the substantially C-shaped second end 66. The substantially C-shaped second end 66 also defines a roof 68 for enclosing at least a portion of the groove 70. The groove 70 and the roof 68 aid in guiding the guide wire 12 into the subdural space 114.

### Statement of Operation

Referring now to FIGS. 5B-8, in order to treat a subdural hematoma, a catheter 56 (see FIGS. 8 and 9) must be partially inserted into the subdural space 114. In order to access the subdural space 114, the surgeon will typically drill a burr hole 120 (there is no burr hole 120 shown in FIG. 8). If using the assembly 10a shown in FIGS. 1-4, 5B-6, and 12-13, the surgeon will then insert the substantially C-shaped second end 26 of the first elongate member 18 into the subdural space 114. Then the surgeon will guide the guide wire 12 substantially downwardly through the bore 36 extending through the shaft portion 34 of the second elongate member 30. The surgeon will continue guiding the guide wire 12 along the groove 28 defined by and extending along the substantially C-shaped second end 26 of the first elongate member 18. Finally, the guide wire 12 will pass into the subdural space 114 in a direction substantially tangent to the curvature of the brain 124.

Once the guide wire 12 is in place, the surgeon will then detach the second elongate member 30 from the groove 54 (or the bore 52) extending along the second side 50 of the housing 46. After detaching the second elongate member 30, the surgeon will then slide it away while the guide wire 12 passes therethrough, while at the same time ensuring that the guide wire 12 maintains its position within the subdural space 114. And finally, the surgeon will remove the substantially C-shaped second end 26 of the first elongate member 18 from the subdural space 114, while at the same time ensuring that the guide wire 12 maintains its position within the subdural space 114. After the assembly 10a has been completely removed, the surgeon will then insert a catheter 56 into the subdural space 114. As shown in FIG. 8 and 9, the guide wire 12 is threaded through the body of the catheter 56. This helps to guide the catheter 56 along the path already created by the guide wire 12. The guide wire 12 is then removed while at the same time ensuring that the catheter 56 maintains its position within the subdural space 114. Drainage of the subdural hematoma may then begin.

If using the assembly 10b of FIGS. 10 and 11, after the burr hole 120 is drilled, the surgeon will insert the substantially C-shaped second end 66 of the elongate member 58 into the subdural space 114. Then the surgeon will guide the guide wire 12 substantially downwardly through the bore 60 extending through the shaft portion 62 of the elongate member 58. The surgeon will continue guiding the guide wire 12 along the groove 70 and under the roof 68 defined by the substantially C-shaped second end 66. Finally, the guide wire 12 will pass into the subdural space 114 in a direction substantially tangent to the curvature of the brain 124. Once the guide wire 12 is in place, the surgeon will remove the substantially C-shaped second end 66 of the elongate member 58 from the subdural space 114 and slide the assembly 10b away while the guide wire 12 passes therethrough, while at the same time ensuring that the guide wire 12 maintains its position within the subdural space 114. After the assembly 10b has been removed, the surgeon will insert the catheter 56. Once the catheter 56 is in position, the guide wire is then removed while at the same time ensuring that the catheter 56 maintains its position within the subdural space 114. Draining of the subdural hematoma is then commenced in the same manner described above.

While the invention has been particularly shown and described with reference to preferred embodiments thereof, it will be understood by those skilled in the art that the foregoing and other changes in form and details may be made therein without departing from the spirit and scope of the invention. For example, although it is preferred that the medical device 14 being inserted through the assembly is a guide wire 12, it should be further understood that substantial benefit may be derived from using the assembly to aid the insertion of a different medical device 14, such as a lead, a catheter, a stent, a filter, a needle, a guide pin, or a cannula. It should be further understood that although it is preferred that the guide wire be made of superelastic nitinol, substantial benefit may be obtained from a guide wire being made of an alternate material, so long as the guide wire may easily maintain its position in the subdural space during removal of the assembly.

I claim:

1. An assembly for guiding insertion of a medical device into a target area of a vertebrate comprising:
a first elongate member, said first elongate member having:
a first end;
a shaft portion coupled to and extending from said first end of said first elongate member; and
a substantially C-shaped second end coupled to and extending from said shaft portion of said first elongate member and disposed opposite said first end of said first elongate member, said substantially C-shaped second end dimensioned to guide said medical device into said target area in a desired direction;
a second elongate member, said second elongate member having:
a first end;
a shaft portion defining a bore extending therethrough and said shaft portion being coupled to and extending from said first end of said second elongate member, said shaft portion dimensioned to permit a medical device to be inserted therethrough; and
a second end coupled to and extending from said shaft portion of said second elongate member and disposed opposite said first end of said second elongate member, said second end of said second elongate member dimensioned to be removably coupled to said substantially C-shaped second end of said first elongate member.

2. The assembly of claim 1 further comprising a housing, said housing dimensioned to be coupled to said second elongate member and said housing dimensioned to be coupled to said first elongate member so that said substantially C-shaped second end of said first elongate member and said second end of said second elongate member form an angle of less than ninety degrees.

3. The assembly of claim 1 further comprising a neck portion coupled to and extending from said shaft portion of said first elongate member.

4. The assembly of claim 1 further comprising a substantially half-cylindrical neck portion coupled to and extending from said shaft portion of said second elongate member.

5. The assembly of claim 1 wherein said second end of said second elongate member being substantially flat.

6. The assembly of claim 1 wherein said vertebrate being a mammal.

7. The assembly of claim 1 wherein said vertebrate being a human being.

8. The assembly of claim 1 wherein said target area being a subdural space.

9. The assembly of claim 8 wherein said desired direction being substantially tangent to a curvature of a brain.

10. The assembly of claim 1 wherein said medical device being at least one of a lead, a guide wire, a catheter, a stent, a filter, a needle, a guide pin, and a cannula.

11. The assembly of claim 1 wherein said medical device being comprised of a shape memory alloy.

12. The assembly of claim 11 wherein said shape memory alloy being a super elastic nitinol alloy.

13. The assembly of claim 1 wherein said substantially C-shaped second end of said first elongate member defining a groove extending along said substantially C-shaped second end of said first elongate member.

14. The assembly of claim 13 wherein said substantially flat second end of said second elongate member providing a roof for enclosing at least a portion of said groove defined by and extending along said substantially C-shaped second end of said first elongate member.

15. The assembly of claim 14 further comprising at least two clasps coupled to and curving substantially inwardly from said roof of said second end of said second elongate member, said clasps being dimensioned to engage said substantially C-shaped second end of said first elongate member.

16. The assembly of claim 2 wherein said housing defining at least two bores extending therethrough, each of said at least two bores dimensioned to receive one of said shaft portion of said first elongate member and said shaft portion of said second elongate member.

17. The assembly of claim 16 wherein said at least two bores being a first bore and a second bore, said first bore extending through said housing proximate a first side of said housing and said second bore extending through said housing proximate a second side of said housing at an approximately forty-five degree angle from said first bore.

18. The assembly of claim 2 wherein said housing defining at least two grooves, each of said at least two grooves dimensioned to snap fit one of said shaft portion of said first elongate member and said shaft portion of said second elongate member.

19. The assembly of claim 18 wherein said at least two grooves being a first groove and a second groove, said first groove extending along a first side of said housing and said second groove extending along a second side of said housing at an approximately forty-five degree angle from said first groove.

20. The assembly of claim 2 wherein said housing defining:
a bore extending therethrough, said bore dimensioned to receive one of said shaft portion of said first elongate member and said shaft portion of said second elongate member; and
a groove, said groove dimensioned to snap fit the other of said shaft portion of said first elongate member and said shaft portion of said second elongate member.

21. The assembly of claim 20 wherein said bore extending through said housing proximate a first side of said housing and said groove extending along a second side of said housing at an approximately forty-five degree angle from said bore.

22. The assembly of claim 1 wherein said medical device having a substantially bulbous insertion end.

23. An assembly for guiding insertion of a medical device into a subdural space comprising:
a first elongate member, said first elongate member having:
a first end;
a shaft portion coupled to and extending from said first end of said first elongate member;
a neck portion coupled to and extending from said shaft portion of said first elongate member;
a substantially C-shaped second end coupled to and extending from said neck portion of said first elongate member and disposed opposite said first end of said first elongate member, said substantially C-shaped second end dimensioned to guide said medical device into said subdural space in a direction substantially tangent to a curvature of a brain; and
a groove defined by and extending along said substantially C-shaped second end of said first elongate member, said groove dimensioned to permit a medical device to pass therethrough;
a second elongate member, said second elongate member having:
a first end;
a shaft portion defining a bore extending therethrough and said shaft portion being coupled to and extending from said first end of said second elongate member, said shaft portion dimensioned to permit a medical device to be inserted therethrough;
a substantially half-cylindrical neck portion coupled to and extending from said shaft portion of said second elongate member;
a substantially flat second end coupled to and extending from said substantially half-cylindrical neck portion of said second elongate member and disposed opposite said first end of said second elongate member, said substantially flat second end providing a roof for enclosing at least a portion of said groove defined by and extending along said substantially C-shaped second end of said first elongate member; and
at least two clasps coupled to and curving inwardly from said roof of said substantially flat second end of said second elongate member, said clasps being dimensioned to engage said substantially C-shaped second end of said first elongate member; and
a housing, said housing defining:
a bore extending therethrough proximate a first side of said housing, said bore dimensioned to receive said shaft portion of said first elongate member; and
a groove extending along a second side of said housing, said groove dimensioned to snap fit said shaft portion of said second elongate member wherein said housing being dimensioned to maintain said second elongate member at an approximately forty-five degree angle to said first elongate member.

24. An assembly for guiding insertion of a medical device into a subdural space comprising an elongate member having:
a first end;
a shaft portion defining a bore extending therethrough and said shaft portion being coupled to and extending from said first end, said shaft portion dimensioned to permit a medical device to be inserted therethrough;
a neck portion coupled to and extending from said shaft portion; and
a substantially C-shaped second end coupled to and extending from said neck portion and disposed opposite said first end, said substantially C-shaped second end dimensioned to guide said medical device into said subdural space in a direction substantially tangent to a curvature of a brain.

25. The assembly of claim 24 wherein said substantially C-shaped second end defining a groove extending along said substantially C-shaped second end, said groove dimensioned to permit a medical device to pass therethrough.

26. The assembly of claim 24 wherein said substantially C-shaped second end defining a roof for enclosing at least a portion of said groove defined by and extending along said substantially C-shaped second end.

27. A method for guiding insertion of a medical device into a target area of a vertebrate comprising, in combination, the steps of:
providing an assembly having:
a first elongate member, said first elongate member having:
a first end;
a shaft portion coupled to and extending from said first end of said first elongate member;
a neck portion coupled to and extending from said shaft portion of said first elongate member;
a substantially C-shaped second end coupled to and extending from said neck portion of said first elongate member and disposed opposite said first end of said first elongate member, said substantially C-shaped second end dimensioned to guide said medical device into said target area in a desired direction; and
a groove defined by and extending along said substantially C-shaped second end of said first elongate member, said groove dimensioned to permit a medical device to pass therethrough;
a second elongate member, said second elongate member having:
a first end;
a shaft portion defining a bore extending therethrough and said shaft portion being coupled to and extending from said first end of said second elongate member;
a substantially half-cylindrical neck portion coupled to and extending from said shaft portion of said second elongate member;
a substantially flat second end coupled to and extending from said substantially half-cylindrical neck portion of said second elongate member and disposed opposite said first end of said second elongate member, said substantially flat second end providing a roof for enclosing at least a portion of said groove defined by and extending along said substantially C-shaped second end of said first elongate member; and
at least two clasps coupled to and curving inwardly from said roof of said substantially flat second end of said second elongate member, said clasps being dimensioned to engage said substantially C-shaped second end of said first elongate member; and
a housing, said housing defining: a bore extending therethrough proximate a first side of said housing, said bore dimensioned to receive said shaft portion of said first elongate member; and
a groove extending along a second side of said housing, said groove dimensioned to snap fit said shaft portion of said second elongate member wherein said housing being dimensioned to maintain said second elongate member at an approximately forty-five degree angle to said first elongate member;
inserting said substantially C-shaped second end of said first elongate member into said target area of said vertebrate;
guiding said medical device substantially downwardly through said bore extending through said shaft portion of said second elongate member;
guiding said medical device along said groove defined by and extending along said substantially C-shaped second end of said first elongate member; and
inserting said medical device into said target area of said vertebrate in said desired direction.

28. The method of claim 27 further comprising the step of inserting said assembly into a subdural space.

29. The method of claim 27 wherein said desired direction being substantially tangent to a curvature of a brain.

30. The method of claim 27 further comprising the steps of:
detaching said second elongate member from said groove extending along said second side of said housing;
sliding said second elongate member away from said target area while said medical device passing therethrough while at the same time said medical device maintaining its position within said target area; and
removing said substantially C-shaped second end of said first elongate member from said target area while at the same time said medical device maintaining its position within said target area.

31. The method of claim 27 wherein said medical device being at least one of a lead, a guide wire, a catheter, a stent, a filter, a needle, a guide pin, and a cannula.

32. The method of claim 27 wherein said medical device comprising a super elastic nitinol alloy.

33. A method for guiding insertion of a super elastic nitinol guide wire into a subdural space comprising, in combination, the steps of:
providing an assembly having:
a first elongate member, said first elongate member having:
a first end;
a shaft portion coupled to and extending from said first end of said first elongate member;
a neck portion coupled to and extending from said shaft portion of said first elongate member;
a substantially C-shaped second end coupled to and extending from said neck portion of said first elongate member and disposed opposite said first end of said first elongate member, said substantially C-shaped second end dimensioned to guide said super elastic nitinol guide wire into said subdural space in a direction substantially tangent to a curvature of a brain; and
a groove defined by and extending along said substantially C-shaped second end of said first elongate member;
a second elongate member, said second elongate member having:
a first end;
a shaft portion defining a bore extending therethrough and said shaft portion being coupled to and extending from said first end of said second elongate member;
a substantially half-cylindrical neck portion coupled to and extending from said shaft portion of said second elongate member;
a substantially flat second end coupled to and extending from said substantially half-cylindrical neck portion of said second elongate member and disposed opposite said first end of said second elongate member, said substantially flat second end providing a roof for enclosing at least a portion of said groove defined by and extending along said substantially C-shaped second end of said first elongate member; and
at least two clasps coupled to and curving inwardly from said roof of said substantially flat second end of said second elongate member, said clasps being dimensioned to engage said substantially C-shaped second end of said first elongate member; and
a housing, said housing defining: a bore extending therethrough proximate a first side of said housing, said bore dimensioned to receive said shaft portion of said first elongate member; and
a groove extending along a second side of said housing, said groove dimensioned to snap fit said shaft portion of said second elongate member wherein said housing being dimensioned to maintain said second elongate member at an approximately forty-five degree angle to said first elongate member;
inserting said substantially C-shaped second end of said first elongate member into said subdural space;
guiding said super elastic nitinol guide wire substantially downwardly through said bore extending through said shaft portion of said second elongate member;
guiding said super elastic nitinol guide wire along said groove defined by and extending along said substantially C-shaped second end of said first elongate member;
inserting said super elastic nitinol guide wire into said subdural space in said direction substantially tangent to said curvature of said brain;
detaching said second elongate member from said groove extending along said second side of said housing;
sliding said second elongate member away from said target area while said super elastic nitinol guide wire passing therethrough while at the same time said super elastic nitinol guide wire maintaining its position within said subdural space; and
removing said substantially C-shaped second end of said first elongate member from said subdural space while at the same time said super elastic nitinol guide wire maintaining its position within said subdural space.

34. A method for guiding insertion of a super elastic nitinol guide wire into a subdural space comprising, in combination, the steps of:
providing an elongate member having:
a first end;
a shaft portion defining a bore extending therethrough and said shaft portion being coupled to and extending from said first end, said shaft portion dimensioned to permit a medical device to be inserted therethrough;
a neck portion coupled to and extending from said shaft portion; and
a substantially C-shaped second end coupled to and extending from said neck portion and disposed opposite said first end, said substantially C-shaped second end defining a groove extending along said substantially C-shaped second end and dimensioned to permit a medical device to pass therethrough, and said substantially C-shaped second end defining a roof for enclosing at least a portion of said groove defined by and extending along said substantially C-shaped second end;
inserting said substantially C-shaped second end into said subdural space;
guiding said super elastic nitinol guide wire substantially downwardly through said bore;
guiding said super elastic nitinol guide wire along said groove;
inserting said super elastic nitinol guide wire into said subdural space in a direction substantially tangent to a curvature of a brain;
removing said substantially C-shaped second end from said target area while said super elastic nitinol guide wire passing therethrough while at the same time said super elastic nitinol guide wire maintaining its position within said subdural space; and
sliding said elongate member away from said target area while said super elastic nitinol guide wire passing therethrough while at the same time said super elastic nitinol guide wire maintaining its position within said subdural space.
